(19) United States
(12) Patent Application Publication
(10) Pub. No.: US 2009/0280518 A1
(43) Pub. Date: Nov. 12, 2009

(54) SYSTEM FOR HIGH THROUGHPUT MEASUREMENT OF MECHANICAL PROPERTIES OF CELLS

(75) Inventors: Andrea Adamo, Cambridge, MA (US); Rangel P. Dokov, Stambolijski (BG); Klavs F. Jensen, Lexington, MA (US)

Correspondence Address: WOLF GREENFIELD & SACKS, P.C., 600 ATLANTIC AVENUE, BOSTON, MA, 02210-2206, US

(73) Assignee: Massachusetts Institute of Technology, Cambridge, MA (US)

(21) Appl. No.: 12/436,814

(22) Filed: May 7, 2009

Related U.S. Application Data
(60) Provisional application No. 61/052,328, filed on May 12, 2008.

(51) Int. Cl. C12Q 1/02; C12M 1/34; G01R 27/22

(52) U.S. Cl. 435/29; 435/287.1; 435/288.7; 324/92

(57) ABSTRACT

A system for measuring a mechanical property of a cell is provided. The system includes a body having a channel therethrough with a first end and a second end, the channel including at least one cell deforming feature configured to deform a cell passing through the channel. A first sensor system is positioned on the first end side of the cell deforming feature and a second sensor system is positioned on the second end side of the cell deforming feature, and the first and second sensor systems are configured to detect information about a cell as the cell travels across the cell deforming feature. A controller communicating with the first and second sensor systems is adapted to receive data from the first and second sensor systems and calculate a mechanical property of the cell.

### RELATED APPLICATIONS

This application claims priority under 35 U.S.C. §119(e) to U.S. Provisional Application Ser. No. 61/052,328, entitled “A SYSTEM FOR HIGH THROUGHPUT MEASUREMENT OF MECHANICAL PROPERTIES OF CELLS” filed on May 12, 2008, which is herein incorporated by reference in its entirety.

### FIELD OF THE INVENTION

The present invention relates to systems and methods for measuring the mechanical properties of cells, and in particular, these systems and methods may be used to determine the mechanical properties of cells to detect various types of disease.

### BACKGROUND OF INVENTION

Recent scientific research has shown a strong correlation between the mechanical properties of a cell and disease. For example, research directed to malaria and urological cancers has shown that the mechanical properties of a diseased cell significantly differ from the mechanical properties of a similar type of healthy cell.

There are various experimental techniques which have been used to measure the mechanical properties of an individual cell, such as micropipette aspiration, optical tweezers/laser traps, magnetic twisting cytometry, atomic force microscopy (AFM indentation), cytoindenter, and fluid shear flow. All of these prior techniques measure cell mechanical properties on a cell by cell basis. Individual cells must be isolated and then separately analyzed to measure the mechanical property of that one cell.

### SUMMARY OF INVENTION

In one aspect, a system for measuring a mechanical property of a cell is provided. The system includes a body having a channel therethrough, where the channel has a first end and a second end. The channel includes at least one cell deforming feature spaced apart from the first end and the second end where the at least one cell deforming feature is configured to deform a cell passing through the channel. The system further includes a first sensor system positioned on the first end side of the at least one cell deforming feature and a second sensor system positioned on the second end side of the at least one cell deforming feature. The first and second sensor systems are configured to detect information about a cell as the cell travels across the cell deforming feature. A controller communicates with the first sensor system and the second sensor system, and the controller is adapted to receive data from the first and second sensor systems and calculate a mechanical property of the cell.

In another aspect, a method of measuring the mechanical properties of a plurality of cells is provided. The method includes introducing a fluid sample into the first end of a channel, where the fluid sample includes a plurality of cells, and detecting information about the plurality of cells as the plurality of cells pass through the channel towards a second end of the channel. The method further includes deforming the plurality of cells, detecting information about the plurality of cells in the channel after the plurality of cells are deformed, and calculating the mechanical properties of the plurality of cells based upon the information detected from the cells.

In yet another aspect, a system for measuring a mechanical property of a cell is provided. The system includes a body having a channel therethrough, the channel having a first end and a second end. The channel includes a constriction in the channel walls spaced apart from the first end and the second end, where the constriction is configured to deform a cell passing through the channel. The system further includes a first sensor system constructed to detect information about a cell in the channel at a position on the first end side of the constriction, and a second sensor system constructed to detect information about a cell in the channel at a position on the second end side of the constriction. A controller communicates with the first sensor system and the second sensor system, where the controller is adapted to receive data from the first and second sensor systems and calculate a mechanical property of the cell.

In another aspect, a system for measuring a mechanical property of a cell is provided. The system includes a body having a channel therethrough, where the channel has a first end and a second end. The channel includes at least one cell deforming feature spaced apart from the first end and the second end where the at least one cell deforming feature is configured to deform a cell passing through the channel. The system further includes a first sensor system constructed to detect information about a cell in the channel at a position on the first end side of the at least one cell deforming feature, and a second sensor system constructed to detect information about a cell in the channel at a position on the second end side of the at least one cell deforming feature. A controller communicates with the first sensor system and the second sensor system, and the controller is adapted to receive data from the first and second sensor systems and calculate a mechanical property of the cell.

### BRIEF DESCRIPTION OF DRAWINGS

The accompanying drawings are schematic and are not intended to be drawn to scale. In the figures, each identical, or substantially similar component that is illustrated in various figures is typically represented by a single numeral or notation. For purposes of clarity, not every component is labeled in every figure, nor is every component of each embodiment of the invention shown where illustration is not necessary to allow those of ordinary skill in the art to understand the invention. In the drawings:

FIG. 1 is a schematic view of a system for measuring the mechanical properties of a cell according to one embodiment of the present invention;

FIGS. 2A-2B are schematic end views of a system for measuring the mechanical properties of a cell according to two embodiments of the present invention;

FIG. 3 is a block diagram for a controller according to one illustrative embodiment;

FIGS. 4A-4D illustrate a cell travelling through a channel according to one illustrative embodiment;

FIGS. 5A-5C illustrate cell deforming features in the channel according to a plurality of different embodiments;

FIGS. 6A-6C illustrate cell deforming features in the channel according to a plurality of different embodiments; and

FIGS. 7A-7B are schematic views of a system for measuring the mechanical properties of a cell according to other embodiments of the present invention.

### DETAILED DESCRIPTION

The present invention provides systems and methods for measuring the mechanical properties of one or more cells. In particular, some aspects of the present invention relate to systems and methods for measuring the mechanical properties of a cell to detect the presence of various types of disease in the cell.

As mentioned above, recent research indicates a strong relationship between the mechanical properties of a cell and whether that cell is healthy or diseased. This research shows that the mechanical properties of a cell, such as its elastic, viscoelastic properties of a cell, and/or spring constants are altered when the cell is diseased. In some instances, a diseased cell is less elastic, less deformable, more fragile, and/or more stiff than a similar cell in a healthy state.

Applicants recognized that the prior experimental techniques used to measure the mechanical properties of a cell are time consuming and expensive. The time and monetary setbacks associated with the current experimental techniques for measuring mechanical properties of a cell make it difficult to realize the benefits of this research and move forward with clinical implementation and the development of useful diagnostic tools. Applicants further recognized that there is a need for a system and/or method for measuring the mechanical properties of a cell that may process and analyze a plurality of cells simultaneously.

Some aspects of the present invention are directed to high throughput systems and methods for measuring mechanical properties of a cell. For example, in one embodiment, a system is provided which is capable of analyzing at least hundreds of cells per second. Such a high throughput system is capable of measuring cells at a much faster rate than the prior experimental techniques. The ability to perform measurements on a large number of cells in a short amount of time may lead to the ability to further exploit the benefits of this recent scientific breakthrough and expand into the clinical and commercial setting. In one embodiment, systems and methods are provided where the mechanical properties of a plurality of cells may be measured simultaneously.

Applicants also recognized that many hospitals current rely on subjective techniques to examine cells. For example, the pathological examination of biopsies is currently a key part of the diagnostic process in a vast majority of hospitals. During a pathological examination, a trained pathologist inspects biological samples and subjectively makes determinations about the cells to make a diagnosis. This process is a non-objective technique that often requires a great deal of time from highly trained physicians.

Thus, aspects of the present invention are directed to a system for measuring the mechanical properties of a cell which may be used as an automated screening tool capable of analyzing biological samples in real time and outputting quantitative data about the mechanical properties of cells to the pathologists and physicians. This data may be less time consuming to attain and/or more reliable than the prior subjective techniques.

The inventive systems and methods for measuring mechanical properties of a cell will now be described in more complete detail in the context of several specific embodiments illustrated in the appended figures. It is to be understood that the embodiments described are for illustrative purposes only and that the inventive features of the invention, as described in the appended claims, can be practiced in other ways or utilized for instruments having other configurations, as apparent to those of ordinary skill in the art.

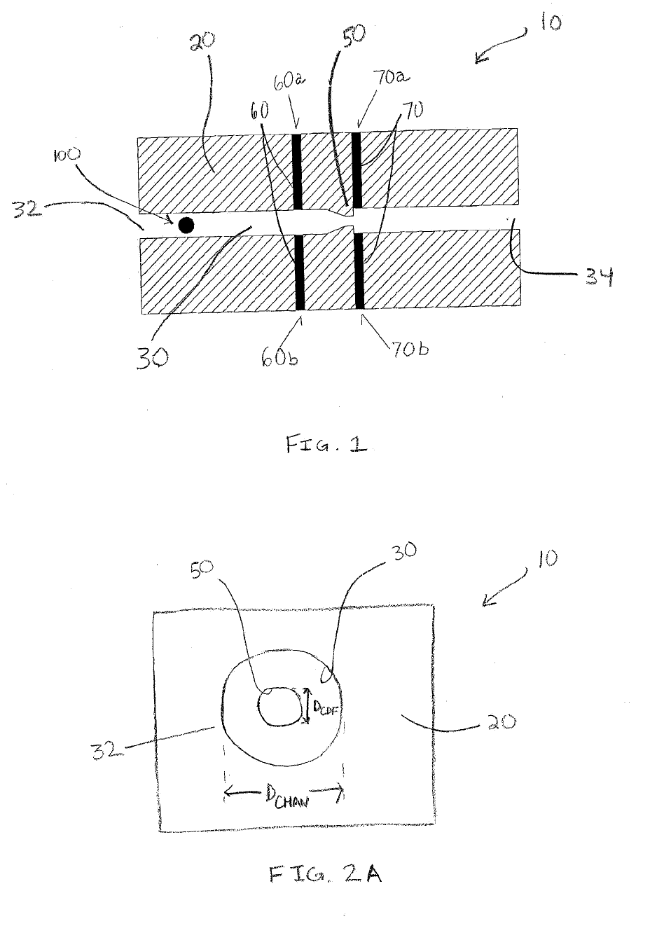

As shown in FIG. 1, in one embodiment, a system 10 is provided which includes a body 20 with a channel 30 extending through the body with the channel 30 having a first end 32 and a second end 34. The channel 30 has a cell deforming feature 50 spaced apart from the first and second ends 32, 34 of the channel 30 which deforms a cell as the cell 100 passes through the channel 30.

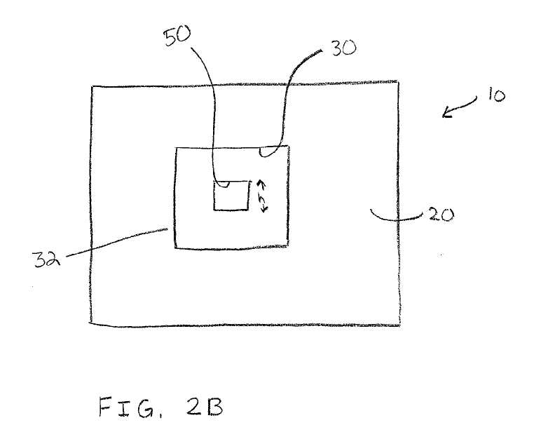

In one embodiment, the cell deforming feature 50 includes a constriction formed by the channel walls which provides a narrow passageway through the channel 30, such that the size of the constriction is less than the size of the channel 30. In one illustrative embodiment shown in FIG. 2A, the cross-section of the channel 30 and the cell deforming feature 50 are circular shaped where the height or diameter DCDF of the cell deforming feature 50 is less than the diameter DCHAN of the channel 30. In another illustrative embodiment shown in FIG. 2B, the cross-section of the channel 30 and the cell deforming feature 50 are rectangular shaped. As will be discussed in greater detail below, the size, shape, and configuration of the channel 30 and cell deforming feature 50 may vary, as the invention is not limited in this respect.

The system 10 for measuring mechanical properties of a cell further includes a a first sensor system 60 positioned on the first end 32 side of the at least one cell deforming feature 50 and a second sensor system 70 positioned on the second end side of the at least one cell deforming feature. The first and second sensor systems 60, 70 are configured to detect information about a cell 100 as the cell travels across the cell deforming feature 50. The sensor systems 60, 70 may be configured to detect information about the cell 100 before, after and/or across the cell deforming feature 50. The first sensor system 60 may be arranged to detect information about a cell 100 in the channel 30 before the cell 100 travels through the cell deforming feature 50. In one embodiment, the first sensor system 60 includes two sensors 60a, 60b where one sensor 60a is positioned within the channel 30 opposite the other sensor 60b. The second sensor system 70 may be arranged to detect information about a cell 100 in the channel after the cell 100 travels through the cell deforming feature 50. In one embodiment, the second sensor system 70 is positioned adjacent the cell deforming feature 50. The second sensor system 70 may also includes two sensors 70a, 70b, where one sensor 70a is positioned within the channel 30 opposite the other sensor 70b.

In one embodiment, one or more of the sensors 60a, 60b, 70a, 70b are electrodes constructed to measure the electrical resistance as the cell 100 travels through the channel 30. For example, the sensors 60a, 60b may include a first electrode and a second electrode positioned on opposite sides of the channel 30 and the sensors 60a, 60b may measure the change in the electrical resistance between the two sensors when the cell 100 travels between the two sensors 60a, 60b. Furthermore, the sensors 70a, 70b on the other side of the cell deforming feature 50 may also include a third electrode and a fourth electrode positioned on opposite sides of the channel 30 and the sensors 70a, 70b may measure the change in the electrical resistance between the two sensors when the cell 100 travels between the two sensors 70a, 70b after it has passed through the cell deforming feature 50.

If the cell 100 is unaltered between the first sensor system 60 and the second sensor system 70, the electrical resistance measured by the second sensor system 70 may be substantially the same as the electrical resistance measured by the first sensor system 60. However, if the cell 100 is altered in some manner between the two sensor systems 60, 70, the measured electrical resistances at each of the sensor systems 60, 70 may be different. This change in electrical resistance may help to quantify this alteration. For example, if the cell deforming feature 50 is configured to deform the cell, the sensor systems 60, 70 may be configured to detect information about how the cell 100 reacts due to being deformed. In one embodiment, the cell deforming feature 50 is configured to be smaller than the size of the cell 100 to be measured, and the cell 100 may be compressed or deformed by the cell deforming feature 50 and the sensor systems 60, 70 may be configured to detect information about how the cell 100 reacts due to being compressed or deformed. It should be recognized that it may be unlikely that the cell will be permanently compressed or deformed.

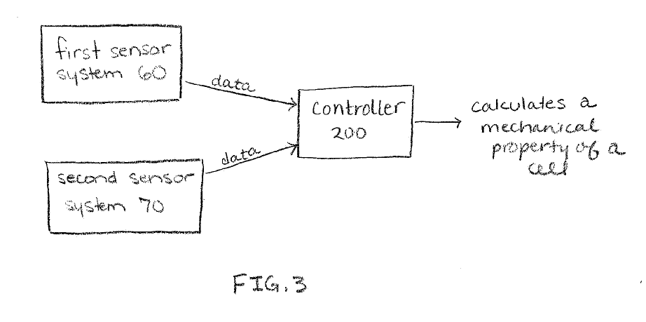

How the cell 100 reacts when compressed or deformed may provide valuable information about mechanical properties of the cell, such as, for example, its elasticity. As shown in FIG. 3, a controller 200 may communicate with the first and second sensor systems 60, 70, and the controller 200 may be adapted to receive the data from the first and second sensor systems 60, 70 to calculate a mechanical property of a cell. The controller 200 may be configured to collect and analyze the information generated by the first and second sensor systems 60, 70, such as electric signals, to determine information about the mechanical properties of the cells.

There are a variety of techniques in which the controller 200 may calculate information that is representative of a mechanical property of a cell. Some of these techniques will be discussed in greater detail below. It should be appreciated that addition techniques may be used in association with the inventive systems and methods discussed herein, as the invention is not limited in this respect.

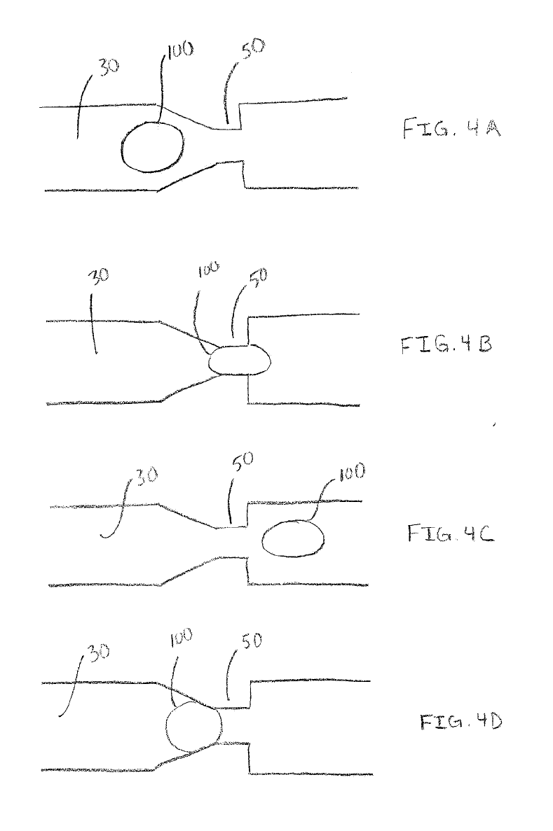

Some techniques to determine the mechanical properties of a cell can be more readily understood when one considers the cell 100 as a sphere of an elastic material that is forced through a narrowing region in a channel that is smaller than the diameter of the sphere-shaped cell 100. As shown in FIG. 4A, the cell 100 may easily pass through the channel 30 when the size of the channel 30 is greater than the size of the cell 100. As shown in FIG. 4B, when the cell 100 approaches a cell deforming feature 50 which is smaller than the size of the cell 100, the cell 100 must compress or deform to pass through the cell deforming feature 50. Once the cell 100 passes through the cell deforming feature 50, it may begin to return to its original shape (FIG. 4C).

The time it takes for the cell 100 to travel through the cell deforming feature 50 may depend upon the elastic properties of the cell. If the cell 100 is more deformable it may flow quickly through the cell deforming feature 50, whereas if the cell 100 is less deformable it may take longer to flow through the cell deforming feature 50, and if the cell is not deformable, it may not be capable of passing through the cell deforming feature 50 and the cell 100 might clog the channel 30 (see FIG. 4D).

Therefore, the more elastic or deformable the cell 100, the faster the cell may travel through the cell deforming feature 50, and thus the shorter the elapsed time it takes for the cell 100 to travel from the first sensor system 60 to the second sensor system 70.

The second sensor system 70 may be arranged to detect information about the cell 100 just after the cell 100 has passed through the cell deforming feature 50. In this respect, the shape of the cell 100 may still be compressed or deformed by the cell deforming feature 50, and the second sensor system 70 may detect information about the shape of the cell 100 and/or the amount the cell changed its shape and/or was deformed by the cell deforming feature 50. If the cell 100 is able to return substantially back to its original shape, the cell 100 may be considered to be relatively elastic and deformable, whereas if the cell 100 does not easily return back to its original shape, the cell 100 may be considered relatively inelastic or less deformable. As mentioned above, a cell 100 which is less elastic and/or deformable may be an indication of a diseased cell.

As mentioned above, the behavior of a cell as the cell passes through a cell deforming feature 50 depends upon the mechanical properties of the cell. In one embodiment of the present invention, the flow of a plurality of cells 100 through the channel 30 and the collection of data from the first and second sensor systems 60, 70 may convey in fractions of a second, a large amount of information about the mechanical properties of the plurality of cells being analyzed substantially simultaneously. For example, in one embodiment, the two sensors 70a, 70b of the second sensor system 70 may detect information on the way a cell 100 behaves when squeezing through the cell deforming feature 50. In one embodiment, the second sensor system 70 is positioned adjacent the cell deforming feature 50 to obtain information about how the cell was affected by the deformation. In one embodiment, the two sensors 70a, 70b may provide information on how the cell 100 appears after the cell deforming feature 50, and/or information on the elastic recovery after deformation. In one embodiment, one sensor 60a, from the first sensor system 60 and one sensor 70b, from the second sensor system 70 may provide information on the travelling time of the cell through the cell deforming feature 50. In one embodiment, the signals between the first and second sensors 60a, 60b of the first sensor system 60 may provide information on the size and diameter of the cell 100 prior to the deformation. This may provide a base line so that appropriate comparisons may be made with the information obtained from the second sensor system 70 about size and diameter of the cell 100 after passing through the cell deforming feature 50.

In some instances, it may be desirable to compare the behavior of one cell relative to other cells of the same type. As mentioned above, the present invention is capable of analyzing a plurality of cells in a substantially simultaneous manner. Certain embodiments provide a system 10 that is able to discriminate among cells that express either variation of the same type of behavior and/or different types of behavior. In other words, in some embodiments, numeric values, such as spring constants, may not be determined. Rather, a system 10 may be used to analyze a sample having a plurality of cells to determine whether there is variation throughout the sample with respect to how the cells 100 react to the same cell deforming feature 50.

To increase the amount of information gathered when a plurality of cells 100 flow through the channel 30 of the system 10, different signals from the electrodes 60a, 60b, 70a, 70b may be collected using AC currents with different frequencies that can be separated when the signal analysis is performed.

Although the above described examples utilize electrodes for the type of sensors 60a, 60b, 70a, 70b for the first and second sensor systems 60, 70, the present invention is not limited in this respect. In other embodiments, it is also contemplated for the first and/or second sensor systems 60, 70 to include other types of sensors, such as, but not limited to various types of electrical sensors, optical sensors, or force sensors. For example, it is contemplated that a force sensor may measure the force acting on the walls of the constriction to provide information on the stiffness of the cell as the cell travels across the constriction. In one embodiment, the first sensor system 60 and/or the second sensor system 70 includes an optical sensing system configured to measure the optical properties as the cell travels across the cell deforming feature.

Furthermore, the present invention is not limited to the configurations of the first and second sensor systems 60, 70 shown in FIG. 1. For example, as shown in FIGS. 7A-7B, it is also contemplated for the first sensor system 60 to include only one sensor, and/or for the second system 70 to only include one sensor. The two sensors may detect information about a cell 100 as the cell 100 travels across the cell deforming feature 50. It should be appreciated that in other embodiments, the first or and/or second sensor systems 60, 70 may include two or more sensors as the invention is not so limited.

As shown in FIG. 2A, in one embodiment, the cross-section of both the channel 30 and the cell deforming feature 50 are circular. As shown in FIG. 2B, in another embodiment, the channel 30 and/or the cell deforming feature 50 has a rectangular shaped cross-section. It should be appreciated that the channel 30 and cell deforming feature 50 may also be formed into other shapes. As illustrated in FIG. 1, in one embodiment, the cell deforming feature 50 has a funnel-shaped region that gradually narrows the channel 50 down to the size and shape of the cell deforming feature 50.

It should be appreciated that the size and shape of the channel 30 and cell deforming feature 50 may vary, as the invention is not limited in this respect. For example, as shown in FIG. 5A, the funnel-shaped narrowing region 52 is longer such that the channel 30 more gradually narrows into the cell deforming feature 50. In contrast, FIG. 5B illustrates another embodiment where there funnel-like narrowing region 52 is relatively shorter, causing the channel 30 to more sharply turn into the cell deforming feature 50. FIG. 5B also illustrates that after the cell deforming feature 50, the channel may also have a funnel-shaped region 54 to allow it to more gradually expand out to the channel 30 diameter. In other embodiments, such as the embodiment illustrated in FIG. 5C, the channel 30 may include a stepped configuration to form the cell deforming feature 50. As shown, the stepped configuration may also be used on the second end 34 side of the cell deforming feature 50 as the channel expands back out to a larger size. It should be appreciated that various slopes and stepped configurations, as well as other configurations may be utilized to transition the channel 30 into the cell deforming feature 50 as the invention is not so limited.

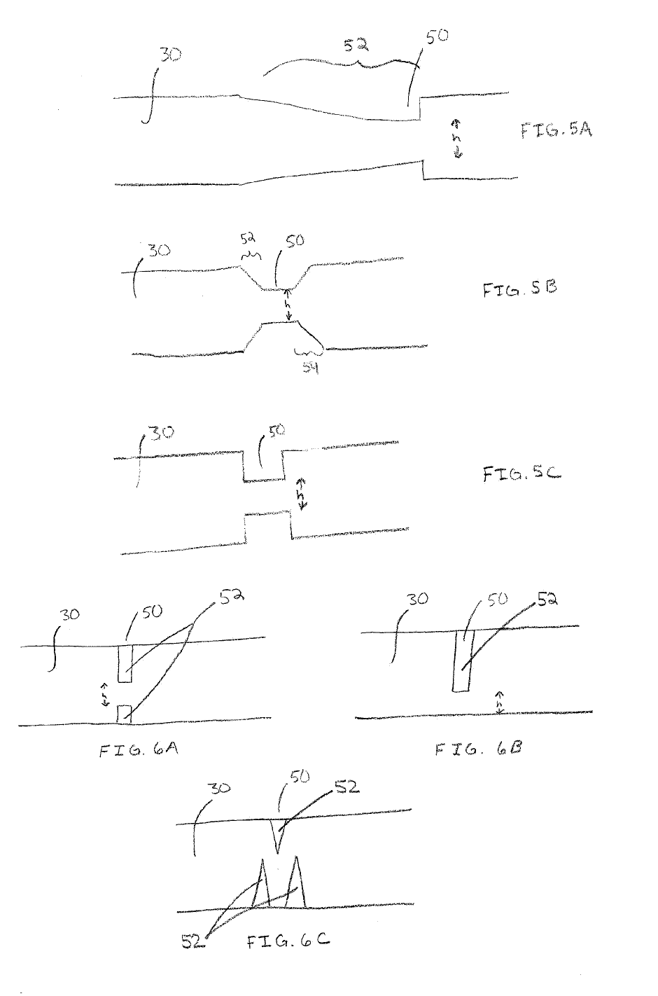

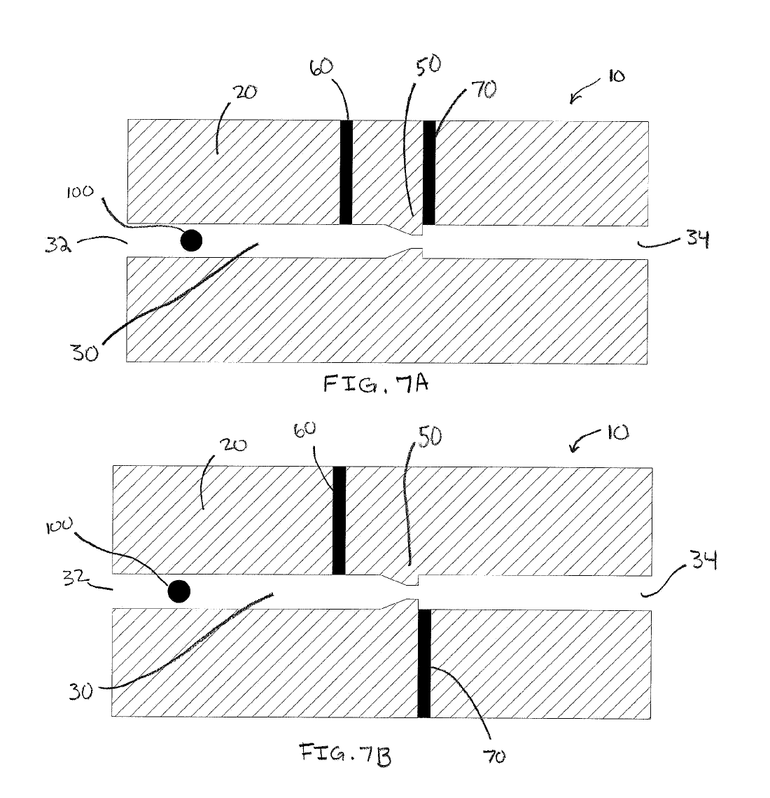

In one embodiment, the cell deforming feature 50 may be formed with at least one obstacle within the channel 30. For example, as shown in FIGS. 6A-6C, the cell deforming feature 50 may include at least one post 52 protruding into the channel 30. As shown in FIG. 6A, in one embodiment, the channel 30 may have at least a first cell deforming feature and a second cell deforming feature which may include a plurality of posts 52. In another embodiment, as shown in FIG. 6B, the cell deforming feature 50 may include only one post 52 extending from the channel walls. As illustrated in FIG. 6C, the cell deforming feature 50 may include a plurality of spaced apart posts 52. It should be appreciated that the cell deforming feature 50 may vary based upon the characteristics of the cell to be measured. For example, in one embodiment the posts 52 may have a substantially flat end as shown in FIGS, 6A-6B, whereas in other embodiments, the posts 52 may have a sharper end, as shown in FIG. 6C.

Although some of the above-mentioned embodiments refer to the diameter of the channel 30 and or the cell deforming feature 50, the present invention is not limited to only circular shaped cross-sections and cylindrical shaped channels 30. It should also be recognized that in other embodiments, the channel 30 and/or the cell deforming feature 50 may be shaped differently, such as, but not limited to square shaped, rectangular, triangular, and/or other geometrical configurations.

It should be appreciated that the size and dimensions of the system 10 may depend upon the size of the cell 100 to be measured. In one embodiment, to deform the cell 100 with a cell deforming feature 50, the size of the cell deforming feature 50 is configured to be smaller in at least one dimension than the size of a cell 100 to be measured. In one embodiment, the height h (See FIGS. 2B, 5-6) of the cell deforming feature 50 is in a range of approximately 2 micrometers to approximately 200 micrometers. In one embodiment, the size of the cell deforming feature 50 is approximately 40% less than the size of the cell 100 to be measured. In another embodiment, the size of the cell deforming feature 50 is approximately 30% less than the size of the cell 100 to be measured. The size of the channel 30 may be in a range of approximately 4 micrometers to approximately 250 micrometers. It should be appreciated that the size of the channel 30 should be large enough to permit the flow of cells 100 through the channel 30. In one embodiment, the distance between the two sensor systems 60, 70 is approximately within the range of approximately 5 micrometers to approximately 1000 micrometers.

In one embodiment, a fluid sample contains a plurality of cells 100 and the fluid sample is introduced into the first end 32 of the channel 30. In one embodiment, the fluid sample may be a concentrated sample of a large number of cells 100. In another embodiment, the fluid sample may be diluted with a fluid to lower the concentration of cells and/or increase the volume of the fluid sample.

Once the fluid sample is introduced into the channel 30, information may be detected about the plurality of cells in the fluid sample as the cells pass through the channel 30 but before the cells 100 travel through the cell deforming feature 50. In one embodiment, the first sensor system 60 may be used to detect this cell information. Once the cell is deformed by the cell deforming feature 50, additional information may be detected about the plurality of cells in the fluid sample. In one embodiment the second sensor system 70 may be used to detect this cell information. In one embodiment, the second sensor system 70 is positioned adjacent the cell deforming feature 50 to obtain information about how the cell was affected by the deformation of the cell deforming feature 50. Thereafter, the mechanical properties of the plurality of cells may be calculated based upon the information detected from the cells.

There are a variety of conventional ways in which the pressure may be created through a channel 30 to move the cells from the first end 32 of the channel 30 through the cell deforming feature 50 and towards the second end 34 of the channel 30. In one embodiment, low pressures, such as those below atmospheric pressure, are used to move the cells. In one embodiment a pump is provided, whereas in other embodiments, it is also contemplated that the cells may be gravity fed through the channel 30. It should be recognized that the flow rate may be selected based upon the particular type of cell to minimize damaging the cell as the cell passes through the cell deforming feature 50.

In embodiments where the pressure within the channel is relatively low, it is feasible to make the system 10 out of a variety of different materials. In one embodiment, the body 20 is made of plastic, but other materials, such as metal, glass, and silicon are also contemplated. It is also contemplated to form this system 10 into a micro-fluidic chip. In one embodiment, the system 10 may be part of a larger system used to process and analyze a cell sample. In one embodiment, a plastic micro-fluidic chip may be designed for disposable use.

The channel 30 may be formed within the body 20 by a variety of techniques. Although the invention is not limited to any particular approach, in one embodiment, the channel 30 is bored out with a laser by known techniques. Furthermore, although the embodiment illustrated in FIG. 2 illustrates a channel that is closed off (other than at the first end 32 and the second end 34), in other embodiments, portions of the channel 30 may be open to the environment as the invention is not so limited. For example, it is contemplated for the channel 30 to be open on one side, as the invention is not so limited.

It should be appreciated that the system 10 may be constructed with any of the common techniques and materials known in the art of microfabrication or used for the fabrication of microfluidic systems. These techniques involve processing steps such as photolithography, wet or dry etching, chemical vapor deposition, wet oxidation, electrodeposition, hot embossing, soft lithography, injection molding, and/or laser ablation. These techniques may utilize various types of materials ranging from, but not limited to, polymers to glass to silicon.

In one embodiment, the cell deforming feature 50 is formed as the channel 30 is formed. In another embodiment, the cell deforming feature 50 may include a separate component which is secured within the channel 30. This separate component may form, for example a post 52, which may be secured within the channel 30 after the channel 30 is formed through the body 20.

The present invention also contemplates systems 10 which may include a heating/cooling element which may communicate with the controller 200 to enable the system 10 to be temperature controlled to allow the mechanical properties of various cells to be measured while the cell is at a controlled temperature. Research has indicated that the mechanical properties of a cell may also vary with temperature and embodiments of the present invention provide ways to measure these properties at the desired temperature.

While several embodiments of the invention have been described and illustrated herein, those of ordinary skill in the art will readily envision a variety of other means and structures for performing the functions and/or obtaining the results or advantages described herein, and each of such variations, modifications and improvements is deemed to be within the scope of the present invention. More generally, those skilled in the art would readily appreciate that all parameters, dimensions, materials, and configurations described herein are meant to be exemplary and that actual parameters, dimensions, materials, and configurations will depend upon specific applications for which the teachings of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described. The present invention is directed to each individual feature, system, material and/or method described herein. In addition, any combination of two or more such features, systems, materials and/or methods, provided that such features, systems, materials and/or methods are not mutually inconsistent, is included within the scope of the present invention. All definitions, as defined and used herein, should be understood to control over dictionary definitions, definitions or usage in documents incorporated by reference, and/or ordinary meanings of the defined terms.

In the claims (as well as in the specification above), all transitional phrases or phrases of inclusion, such as “comprising,” “including,” “carrying,” “having,” “containing,” “composed of,” “made of,” “formed of,” “involving” and the like shall be interpreted to be open-ended, i.e. to mean “including but not limited to” and, therefore, encompassing the items listed thereafter and equivalents thereof as well as additional items. Only the transitional phrases or phrases of inclusion “consisting of” and “consisting essentially of” are to be interpreted as closed or semi-closed phrases, respectively. The indefinite articles “a” and “an,” as used herein in the specification and in the claims, unless clearly indicated to the contrary, should be understood to mean “at least one.”

What is claimed is:

1. A system for measuring a mechanical property of a cell, the system comprising:
a body having a channel therethrough, the channel having a first end and a second end, wherein the channel includes at least one cell deforming feature spaced apart from the first end and the second end, wherein the at least one cell deforming feature is configured to deform a cell passing through the channel;
a first sensor system positioned on the first end side of the at least one cell deforming feature and a second sensor system positioned on the second end side of the at least one cell deforming feature, wherein the first and second sensor systems are configured to detect information about a cell as the cell travels across the cell deforming feature; and
a controller communicating with the first sensor system and the second sensor system, wherein the controller is adapted to receive data from the first and second sensor systems and calculate a mechanical property of the cell.

2. The system of claim 1, wherein the first sensor system includes a first electrode and a second electrode configured to measure the electrical resistance as the cell travels through the channel adjacent the first and second electrodes.

3. The system of claim 2, wherein the first electrode is positioned within the channel opposite the second electrode.

4. The system of claim 2, wherein the second sensor system includes a third electrode and a fourth electrode configured to measure the electrical resistance as the cell travels through the channel adjacent the third and fourth electrodes.

5. The system of claim 4, wherein the third electrode is positioned within the channel opposite the fourth electrode.

6. The system of claim 1, wherein the first and second sensor systems include an optical sensing system configured to measure the variation of optical qualities as the cell travels across the cell deforming feature.

7. The system of claim 1, wherein at least one of the first sensor system and the second sensor system includes an optical sensing system configured to measure the variation of optical qualities as the cell travels across the cell deforming feature.

8. The system of claim 1, wherein the second sensor system is positioned adjacent the at least one cell deforming feature.

9. The system of claim 1, wherein the at least one cell deforming feature includes a constriction formed by the channel walls.

10. The system of claim 1, wherein the at least one cell deforming feature includes a funnel-shaped region.

11. The system of claim 1, wherein the perimeter of the at least one cell deforming feature is rectangular shaped.

12. The system of claim 1, wherein the perimeter of the channel is rectangular shaped.

13. The system of claim 1, wherein the at least one cell deforming feature includes at least one post protruding into the channel.

14. The system of claim 1, wherein the at least one cell deforming feature includes at least a first cell deforming feature and a second cell deforming feature.

15. A method of measuring the mechanical properties of a plurality of cells comprising the steps of:
introducing a fluid sample into the first end of a channel, wherein the fluid sample includes a plurality of cells;
detecting information about the plurality of cells as the plurality of cells pass through the channel towards a second end of the channel;
deforming the plurality of cells;
detecting information about the plurality of cells in the channel after the plurality of cells are deformed; and
calculating the mechanical properties of the plurality of cells based upon the information detected from the cells.

16. The method of measuring the mechanical properties of a plurality of cells according to claim 15, wherein the information about the plurality of cells is detected with a plurality of electrodes configured to measure the electrical resistance as the cells travel through the channel.

17. The method of measuring the mechanical properties of a plurality of cells according to claim 15, wherein the information about the plurality of cells is detected with an optical sensing system configured to measure the optical properties as the cells travel through the channel.

18. The method of measuring the mechanical properties of a plurality of cells according to claim 15, wherein the plurality of cells are deformed with a constriction formed by the channel walls.

19. A system for measuring a mechanical property of a cell, the system comprising:
a body having a channel therethrough, the channel having a first end and a second end, wherein the channel includes a constriction in the channel walls spaced apart from the first end and the second end, where the constriction is configured to deform a cell passing through the channel;
a first sensor system constructed and arranged to detect information about a cell in the channel at a position on the first end side of the constriction;
a second sensor system constructed and arranged to detect information about a cell in the channel at a position on the second end side of the constriction; and
a controller communicating with the first sensor system and the second sensor system, wherein the controller is adapted to receive data from the first and second sensor systems and calculate a mechanical property of the cell.

20. A system for measuring a mechanical property of a cell, the system comprising:
a body having a channel therethrough, the channel having a first end and a second end, wherein the channel includes at least one cell deforming feature spaced apart from the first end and the second end, wherein the at least one cell deforming feature is configured to deform a cell passing through the channel;
a first sensor system constructed and arranged to detect information about a cell in the channel at a position on the first end side of the at least one cell deforming feature;
a second sensor system constructed and arranged to detect information about a cell in the channel at a position on the second end side of the at least one cell deforming feature; and
a controller communicating with the first sensor system and the second sensor system, wherein the controller is adapted to receive data from the first and second sensor systems and calculate a mechanical property of the cell.
